(19) United States
(12) Patent Application Publication
(10) Pub. No.: US 2010/0048941 A1
(43) Pub. Date: Feb. 25, 2010

(54) BIODIESEL PROCESS AND CATALYST THEREFOR

(75) Inventors: Louis A. Kapicak, Cross Lanes, WV (US); David James Schreck, Lake City, MN (US)

Correspondence Address: PAULEY PETERSEN & ERICKSON, 2800 WEST HIGGINS ROAD, SUITE 365, HOFFMAN ESTATES, IL, 60169, US

(73) Assignee: BEST ENERGIES, INC., Madison, WI (US)

(21) Appl. No.: 12/531,446

(22) PCT Filed: Mar. 14, 2008
(86) PCT No.: PCT/US2008/057041

§ 371 Date: Sep. 15, 2009

Related U.S. Application Data
(60) Provisional application No. 60/918,514, filed on Mar. 16, 2007.

(51) Int. Cl. C07C 69/02; B01J 31/02

(52) U.S. Cl. 560/231; 502/171

(57) ABSTRACT

Basic metal salt of glycerin is used as transesterification catalyst or an intermediate to an anhydrous transesterification catalyst for the base catalyzed process for making biodiesel from fats and oils.

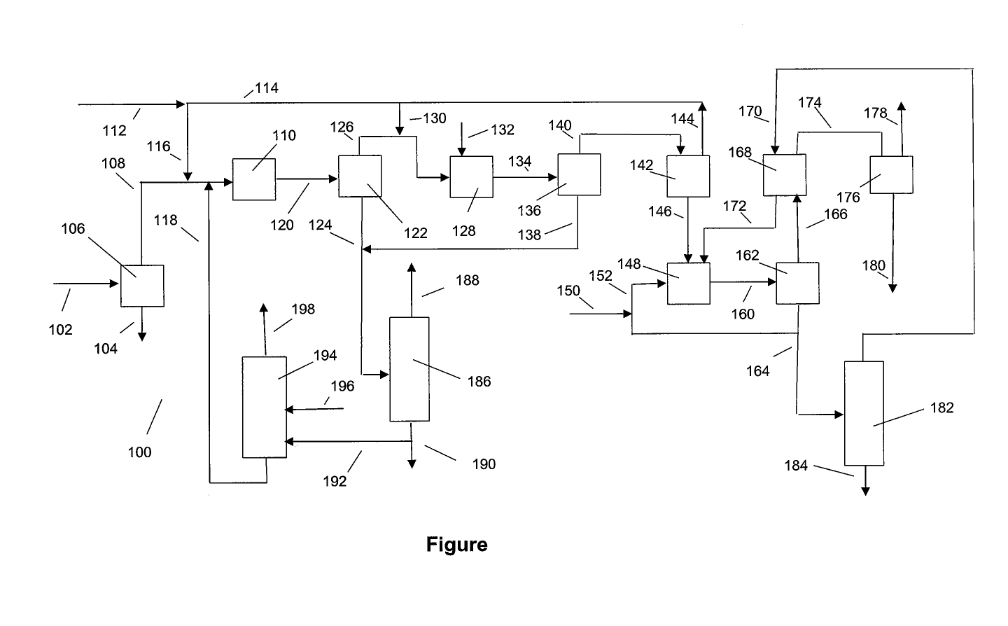

### FIELD OF THE INVENTION

This invention pertains to processes for the synthesis of biodiesel from fats and oils by base catalyzed transesterification with lower alkanol, and particularly to such processes in which base catalyst is directly or indirectly provided by basic metal salt of glycerin. In preferred aspects of the invention, the processes for the synthesis of biodiesel are integrated with the preparation of substantially anhydrous basic metal salt of glycerin.

### BACKGROUND TO THE INVENTION

Biodiesel is being used as an alternative or supplement to petroleum-derived diesel fuel. Biodiesel can be made from various bio-generated oils and fats from vegetable and animal sources.

One process involves the transesterification of triglycerides in the oils or fats with a lower alkanol in the presence of a catalyst to produce alkyl ester useful as biodiesel and a glycerin co-product. In this process, the alkyl ester and glycerin are separated, usually by a phase separation, and the lighter phase containing crude biodiesel is refined. Typically refining operations include the removal of residual alkanol, glycerin and other impurities present in the crude biodiesel.

While the catalyst for the transesterification may be a basic or acidic catalyst, the use of a basic catalyst is often preferred since the transesterification conditions are generally milder than those required for equivalent conversion rates using acidic catalysts such as sulfuric acid. Typically alkali metal hydroxide or alkoxide of the lower alkanol is used as the basic catalyst. The alkali metal is often sodium.

A concern in the synthesis of biodiesel is the yield of biodiesel based upon the triglyceride contained in the fat or oil used as feedstock. Water present during the transesterification can react with a glyceride to form a carboxylic acid instead of the sought lower alkanol ester, which in turn reacts with the basic catalyst to form soap. Thus not only is a loss in yield incurred but also catalyst is lost. To reduce water concentration during the transesterification, operators have chosen to use substantially anhydrous alkali metal hydroxide, or even more preferably, use a substantially anhydrous lower alkoxide of the alkali metal. The need to remove water results in higher costs for such anhydrous catalysts. Even when, e.g., a sodium alkoxide catalyst is sought, the reaction between sodium hydroxide and the lower alkanol, is equilibrium limited such that water formed by the reaction between the hydroxide and lower alkanol must be driven off. Due to the lower boiling point of methanol or the azeotropes formed by ethanol and propanol with water, providing the sought alkoxide with removal of water involves a significant effort.

Accordingly, a need exists for relatively inexpensive sources of basic metal catalysts for the base catalyzed synthesis of biodiesel.

### SUMMARY OF THE INVENTION

By this invention processes are provided for the synthesis of biodiesel from oils and fats by base catalyzed transesterification with lower alkanol using a basic metal salt of glycerin to provide the catalyst or an intermediate to an anhydrous catalyst. Due to the much higher boiling point of glycerin than that of water, water can readily be removed thereby facilitating conversion to the glycerin salt and providing a glycerin salt of desirably low water content. The anhydrous glycerin salt may be introduced directly into the reaction menstruum for the transesterification, or it can be pre-reacted with lower alkanol to provide a basic metal lower alkoxide which can serve as catalyst.

A further advantage of the processes of this invention using a basic metal salt of glycerin is that considerable flexibility exists in the source of the basic metal. For instance, aqueous solutions of reactive basic metal compounds such as hydroxide, oxides or carbonates can be used due to the ease and relatively low cost of water removal. In preferred aspects of the processes of this invention, the glycerin for preparing the salt of glycerin is generated from the transesterification of the oils or fats. Hence, if desired, the catalyst preparation can be integral with the biodiesel synthesis process in which glycerides are converted to lower alkyl esters and glycerin.

In one broad aspect of the processes of this invention for the base catalysis of glycerides to form lower alkyl esters comprises contacting said glyceride with lower alkanol under base catalyzed transesterification conditions comprising the presence of a catalytically effective amount of base catalyst comprising basic metal salt of glycerin to form monoalkyl ester and glycerin. Basic metals comprise one or more of alkali metals and alkaline earth metals, preferably at least one of sodium, potassium and calcium, and most preferably at least one of sodium and potassium. Preferably the base catalyst contains less than about 1, more preferably less than about 0.2, and most preferably less than about 0.05, mole of water per mole of glycerin salt. While the catalyst is referred to herein as a glycerin salt, it is to be understood that the catalyst is introduced as the glycerin salt and the catalytically active species may comprise a reaction product of the glycerin salt in the transesterification menstruum.

Another broad aspect of the processes of this invention for the base catalysis of glycerides to form lower alkyl esters comprises
a. contacting glyceride with lower alkanol under base catalyzed transesterification conditions comprising the presence of a catalytically effective amount of base catalyst to form monoalkyl ester and glycerin;
b. separating monoalkyl ester and glycerol to provide a monoalkyl ester fraction and a glycerin fraction;
c. in a separate step, contacting at least a portion of the glycerin fraction with at least one basic metal compound with glycerin and removing by vaporization water to provide a basic metal salt of glycerin, preferably containing less than about 1, more preferably less than about 0.2, and most preferably less than about 0.05, mole of water per mole of glycerin salt; and
d. using at least a portion of the basic metal salt of glycerin to provide at least a portion of the base catalyst for step a.

The invention also relates in yet another broad aspect to a process for making basic transesterification catalyst comprising reacting at least one basic metal compound with glycerin to form metal salt of glycerol and removing by vaporization water. Preferably the catalyst contains less than about 1, more preferably less than about 0.2, and most preferably less than about 0.05, mole of water per mole of glycerin salt. In a preferred embodiment of this aspect of the invention, the glycerin salt is substantially anhydrous, preferably containing less than about 1, more preferably less than about 0.5, and most preferably less than about 0.2, mass percent water and is reacted with lower alkanol to provide basic metal alkoxide which comprises at least a portion of the transesterification catalyst.

Another broad aspect of this invention relates to transalkylation catalyst comprising basic metal salt of glycerin. Preferably the catalyst contains less than about 1, more preferably less than about 0.2, and most preferably less than about 0.05, mole of water per mole of glycerin salt. Another transalkylation catalyst of this invention comprises at least about 5, preferably at least about 10, mass percent lower alkoxide of basic metal and glycerin wherein the mole ratio of glycerin to lower alkoxide is less than about 5:1. Preferably this transalkylation catalyst contains less than about 0.2 mole of water per mole of lower alkoxide of basic metal. Preferably, the lower alkanol comprises one or more of methanol, ethanol, n-propanol, i-propanol, 1-butanol, isobutanol and 2-butanol.

### BRIEF DESCRIPTION OF THE DRAWING

The FIGURE is a schematic depiction of an apparatus for performing the processes of this invention wherein transesterification catalyst is made using glycerin coproduced in making biodiesel.

### DETAILED DISCUSSION

### The Catalyst

The transesterification catalyst or catalyst precursor of this invention comprises a basic metal salt of glycerin. The glycerin salt may be used as the catalyst or may be further reacted with lower alkanol to provide a basic metal alkoxide. The basic metal alkoxide may be recovered or used as a mixture containing the basic metal alkoxide and glycerin. The reaction with lower alkanol may be prior to introduction into the transesterification zone or may occur in whole or part in the transesterification zone.

The basic metal salt of glycerin may be made in any convenient manner. Usually the salt is prepared by reacting glycerin with one or more of reactive basic metal compounds such as oxides, hydroxides, bicarbonates or carbonates. Basic metals include alkali and alkaline earth metals such as sodium, potassium, lithium, cesium, barium, calcium and strontium. Due to availability and cost, sodium, potassium and calcium are the preferred basic metals. The reactive basic metals compounds are those that will react with glycerin to form the glycerin salt. Examples of basic metal compounds include, but are not limited to sodium hydroxide, sodium carbonate, sodium bicarbonate, potassium hydroxide, potassium oxide, potassium carbonate, potassium bicarbonate, calcium hydroxide, calcium oxide, calcium bicarbonate and calcium carbonate. The basic metal compounds may be supplied in a relatively pure form or may be admixed with other components. These compounds may be solid or dissolved or suspended in a solution. Where in solution or suspension, a protic solvent may be used, e.g., an aqueous or alcohol or ether solution or suspension exists. One of the advantages of the invention is that due to the higher boiling point of glycerin, water can readily be removed by selective vaporization during the formation of the salt. Hence, relatively inexpensive sources of basic metal compounds such as aqueous sodium or potassium hydroxides, potash, and lime, are useful.

Any suitable source of glycerin can be used. For instance, the glycerin may be reagent grade glycerin, but most conveniently it is derived from the biodiesel synthesis process itself. Again, the presence of water poses no undue problem as the water can be removed by vaporization. In typical biodiesel processes, phase separation of transesterification menstruum is used to provide crude biodiesel-containing fraction and a heavier, glycerin-containing fraction. This glycerin-containing fraction typically contains some lower alkanol and soaps, if present. In some instances, the glycerin-containing layer obtained from the transesterification may be used as is, or the glycerin-containing layer may be refined. In either event, it is desirable that the soap content of the glycerin-containing feed be less than about 3, preferably less than about 2.5, mass percent as soaps can cause the formation of foams that make more complex the process for making the glycerin salt. In the preferred integrated processes of this invention, the water content of the glycerin salt is less than about 0.2 mass percent such that the formation of soaps due to water in the catalyst is attenuated. This low water content, in return, enhances the ability to have a glycerin-containing fraction having the desired, low soap content.

The mole ratio of basic metal to glycerin for preparing the glycerin salt may vary over a wide range. Since glycerin is contained in the transesterification reaction menstruum, there is little disadvantage in using a stoichiometric excess of glycerin. The mono-, di- and/or tri-salt of glycerin may be formed. Usually the mono-salt of the glyceride is sufficient to provide adequate catalytic activity. Typically the ratio of basic metal atoms to moles of glycerin used for making the glycerin salt is within the range of between about 0.001:1 to 5:1, preferably between about 0.01 to 3:1, and most often between about 0.1 to 1:1, say, 0.5:1 to 0.9:1.

The preparation of the glycerin salt may be continuous, semi-continuous or batch. The reaction conditions for making the glycerin salt will vary widely depending upon, among other things, the type of basic salt compound used as well as the desired conversion of the basic salt compound to glycerin salts, the glycerin salts being formed and the degree of water removal from the glycerin salts. Often the temperature for the salt formation is within the range of about 10° to 200° C., say, 15° to 170° C. Higher temperatures are preferred, not only to favor the removal of water, but also to reduce the viscosity of glycerin to facilitate its handling. The reaction may be conducted at any suitable pressure, e.g., from about 0.1 to 500 kPa absolute or more. The duration of the reaction may also vary widely depending upon other reaction conditions and the extent of conversion to the glycerin salt that is sought. Often the duration is from about 0.01 to 50 or more hours.

For some glycerin salts, such as a salt of calcium, a precipitate is formed, whereas for sodium or potassium, the glycerin salt product has some solubility in glycerin. If a liquid product containing glycerin salt is desired, the use of an excess of glycerin as well as higher temperatures may be preferable. Preferably periodically or continuously water is removed from the reaction menstruum to assist in driving the reaction toward the formation of the glycerin salt. Where water is removed during the reaction to form the glycerin salt, preferably subatmospheric pressure is used, e.g. from about 0.1 to 90, more preferably from about 1 to 50, kPa absolute, although atmospheric and higher pressures are still operable.

Where a precipitate of the glycerin salt occurs, the precipitate may be recovered prior to any water removal. Where an excess of glycerin is used to maintain a substantially liquid product containing glycerin salt, the mole ratio of free glycerin to glycerin salt is often in the range of about 0.5:1 to 5:1 or 10:1 or more. If the free glycerin is not removed from the liquid product, it is generally preferred that the mole ratio of glycerin to glycerin salt be less than about 5:1.

The extent of water removal can also be within a broad range. Where soap formation in the transesterification process is sought to be minimized, water content of the glycerin salt is preferably relatively low, e.g., less than about 2, preferably less than about 0.5, and most preferably less than about 0.2, mass percent based on the mass of the product containing the glycerin salt. More water can be tolerated in products containing the glycerin salt that also contain significant amounts of glycerin due to the hygroscopic nature of glycerin.

The glycerin salt-containing product may be used directly in the transesterification to provide catalyst, or all or a portion may be reacted with lower alkanol. Preferably the lower alkanol comprises one or more of methanol, ethanol, n-propanol, i-propanol, 1-butanol, isobutanol and 2-butanol. Where the glycerin salt is a solid, the lower alkanol, in some instances, can dissolve or suspend the salt and can react with the solid glycerin salt to form the lower alkoxide of the base metal. Where the glycerin salt is pre-reacted, the product containing glycerin salt is contacted with lower alkanol under conditions sufficient to provide the lower alkoxide of the basic metal. The lower alkoxide of the basic metal can be separated from the glycerin or, more often, a mixture is formed. This mixture of lower alkoxide and glycerin can be introduced into the transesterification menstruum. In the pre-reaction, the mole ratio of glycerin salt to lower alkanol is typically in the range of from about 1:100 to 100:1. The temperature may be in the range of from about 10° to 200° C., preferably from about 20° to 80° C. The duration of the reaction is often from about 0.01 to 50 hours or more. In some instances it may be desired to form the lower alkoxide at higher temperatures to maintain the glycerin salt in a liquid or as a solute in glycerin and lower alkanol. These higher temperatures may require the use of elevated pressures.

### The Biodiesel Synthesis Process

The following discussion is in reference to the facility depicted in the FIGURE. The FIGURE is not intended to be in limitation of this invention. Reference is made herein to copending PCT/US07/20248, herein incorporated by reference in its entirety for certain processes for making biodiesel.

With respect to the FIGURE, biodiesel manufacturing facility 100 uses a suitable raw material feed provided via line 102. The feed may be one or more suitable oils or fats derived from bio sources, especially vegetable oils and animal fats. Examples of fats and oils are rape seed oil, soybean oil, cotton seed oil, safflower seed oil, castor bean oil, olive oil, coconut oil, palm oil, corn oil, canola oil, fats and oils from animals, including from rendering plants and fish oils. The oils and fats may contain free fatty acids falling within a broad range. Generally, the free fatty acid in the raw material feed is less than about 60, and unless pretreatment occurs to remove free fatty acids, preferably less than about 10, mass percent (dry basis). The balance of the fats and oils is largely fatty acid triglycerides. The unsaturation of the free fatty acids and triglycerides may also vary over a wide range. Typically, some degree of unsaturation is preferred to reduce the propensity of the biodiesel to gel at cold temperatures.

As shown, the raw material feed in line 102 is passed to pretreatment unit 106 which may effect one or more unit operations to enhance the feed as a transesterification feedstock such as drying, free fatty acid removal, filtration to remove particulates, and the like. Line 104 shows a discharge of rejected material from such unit operations.

A glyceride-containing feed is passed from unit operations 106 via line 108 to reactor 110 for transesterification. The transesterification is a catalyzed reaction with a lower alkanol, preferably methanol, ethanol or isopropanol wherein the catalyst comprises catalyst in accordance with this invention. Higher alkanols can be used. Methanol is the most preferred alkanol not only due to its availability but also because of its ease of recovery by vapor fractionation. For purposes of the following discussion, methanol will be the alkanol.

As shown, methanol is supplied via line 112 to methanol header 114. Line 116 supplies methanol to reactor 110. Although line 116 is depicted as introducing methanol into line 108, it is also contemplated that methanol can be added directly to reactor 110. Generally methanol is supplied only in a slight excess above that required to achieve the sought degree of transesterification in reactor 110. More methanol can be supplied but it may be lost from the facility. Preferably, the amount of methanol is from about 101 to 500, more preferably, from about 105 to 200, mass percent of that required for the sought degree of transesterification in reactor 110. In the facility depicted, two reactors are used. One reactor may be used, but since the reaction is equilibrium limited, most often at least two reactors are used. Often, where more than one reactor is used, at least about 60, preferably between about 70 and 96, percent of the glycerides in the feed are reacted in the first reactor.

The base catalyst is shown as being introduced via line 118 to reactor 110. Preferably, the amount of catalyst is from about 101 to 200, more preferably, from about 101 to 150, mass percent of that required for the sought degree of transesterification in reactor 110.

The transesterification in reactor 110 is often at a temperature between about 30° C. and 220° C., preferably between about 30° C. and 80° C. The pressure is typically in the range of between about 90 to 500 kPa (absolute) although higher and lower pressures can be used. The reactor is typically batch, semi-batch, plug flow or continuous flow tank with some agitation or mixing, e.g., mechanically stirred, ultrasonic, static mixer, e.g., a packed bed, baffles, orifices, venturi nozzles, tortuous flow path, or other impingement structure. The residence time will depend upon the desired degree of conversion, the ratio of methanol to glyceride, reaction temperature, the degree of agitation and the like, and is often in the range of about 0.1 to 20, say, 0.5 to 10, hours.

The partially transesterified effluent for reactor 110 is passed via line 120 to phase separator 122. Phase separator 122 may be of any suitable design and provides a glycerin-containing bottoms stream passed via line 124. The material in line 124 can be subjected to suitable unit operations to recover components thereof. This stream also contains any soaps made in reactor 110 and a portion of the catalyst. The lighter phase contains alkyl esters and unreacted glycerides and is passed via line 126 to second transesterification reactor 128.

Reactor 128 may be of any suitable design and may be similar to or different than reactor 110. As shown, additional methanol is provided via line 130 from methanol header 114 and additional catalyst is provided via line 132. Preferably the transesterification conditions in reactor 128 are sufficient to react at least about 90, more preferably at least about 95, and sometimes at least about 97 to 99.9, mass percent of the glycerides in the feed to reactor 110. The transesterification in reactor 128 is typically operated under conditions within the parameters set forth for reactor 110 although the conditions may be the same or different. The residence time will depend upon the desired degree of conversion. Typically, it is desired that the conversion be at least about 98, preferably at least about 99, percent complete based upon the conversion of the glycerides in the feed.

The effluent from reactor 128 is passed via line 134 to phase separator 136 which may be of any suitable design and may be the same as or different from the design of separator 122. A heavier, glycerine-containing phase is withdrawn via line 138. This stream contains some catalyst and methanol. A lighter phase containing crude biodiesel is withdrawn from separator 136 via line 140. The lighter phase also contains catalyst and methanol.

The crude is then passed to methanol separator 142. Preferably the catalyst is neutralized with acid prior to being introduced into methanol separator 142. Methanol separator 142 effects a fast, vapor fractionation of the lower alkanol from the crude biodiesel and may be of any convenient design including a stripper, wiped film evaporator, falling film evaporator, and the like. Where subatmospheric pressure is used, it is preferred to use a liquid ring vacuum pump.

As stated above, a falling film evaporator is the preferred apparatus for effecting the vapor fractionation. The tubes of the falling film evaporator may be circular in cross section or any other convenient cross-sectional shape, and the tubes may have a constant cross-sectional configuration over their length or may be tapered or otherwise change in cross-sectional configuration.

Often the vapor fractionation recovers at least about 70, preferably at least about 90, mass percent of the lower alkanol contained in the crude biodiesel. Any residual alkanol is substantially removed in any subsequent water washing of the crude biodiesel. Advantageously, the amount of alkanol contained in the spent water from the washing may be at a sufficiently low concentration that the water can be disposed without further treatment. However, from a process efficiency standpoint, methanol can be recovered from the spent wash water for recycle to the transesterification reactors.

The lower boiling fraction containing the lower alkanol will contain a portion of any water contained in the crude biodiesel. Since the transesterification is conducted with little water being present, and a portion of the water is removed with the glycerin, the concentration of water in this fraction can be sufficiently low that it can be recycled to the transesterification reactors. This lower boiling fraction often contains less than about 0.1, and more preferably less than about 0.05, mass percent water. The methanol-containing fraction is removed from separator 142 via line 144 and may be exhausted from the facility as a waste stream, e.g., for burning or other suitable disposal, or can be added to the methanol header 114.

The methanol separation preferably lowers the lower alkanol content of the bottoms stream to less than about 10, more preferably less than about 2, milligrams of lower alkanol per kilogram of alkyl ester in the bottoms stream. If the catalyst has not been previously neutralized, the bottoms stream from methanol separator 142 is contacted with an aqueous acid solution to neutralize the catalyst.

As shown, the bottoms stream is subjected to a strong acid treatment to recover free fatty acids. Often, if only base catalyst neutralization is sought, a much weaker and smaller volume acid solution can be used.

The bottoms stream is passed via line 146 to mixer 148. Into mixer 148 is passed a strong acid aqueous solution via line 152. Mixer 148 may be an in-line mixer or a separate vessel. Mixer 148 should provide sufficient mixing and residence time that essentially all of the soaps are converted to free fatty acids. Often the temperature during the mixing is in the range of about 80° C. to 220° C., and for a residence time of between about 0.01 to 4, preferably 0.02 and 1, hours.

In accordance with the processes of this invention, the strong acid aqueous solution introduced via line 152 has a pH sufficient to convert the soaps to free fatty acids. Often the pH is less than about 5, sometimes less than about 4, and more preferably less than about 3, say, between about 0.1 and 2.5. The acid may be any suitable acid to achieve the sought pH such as hydrochloric acid, sulfuric acid, sulfonic acid, phosphoric acid, perchloric acid and nitric acid. Sulfuric acid is preferred due to cost and availability. The amount of strong acid aqueous solution provided is typically in a substantial excess of that required to convert the soaps to free fatty acid and to neutralize any remaining catalyst. The excess of acid is often at least about 5, preferably at least about 10, say between about 10 and 1000 times that required. Consequently the effluent from mixer 148 is at a pH of up to about 4, preferably between about 0.1 and 3.

The effluent from mixer 148 is passed via line 160 to phase separator 162. Phase separator 162 may be of any suitable design. A lower aqueous phase is withdrawn via line 164 for distillation. If desired, a portion of this aqueous phase can be recycled via line 152 to mixer 148. Make-up acid is provided via line 150 to line 152. Alternatively, make-up acid can be added to line 172, described below and no recycle 152 need be employed.

The lighter phase which contains crude biodiesel and free fatty acid is withdrawn via line 166 and is passed to water wash column 168. Fresh water enters column 168 via line 170 and serves to remove residual methanol and salts from the crude biodiesel. Normally the column is operated at a temperature between about 20° C. and 80° C., preferably between about 35° C. and 75° C. In a preferred embodiment, the spent water from wash column 168 is passed via line 172 to mixer 148 or combined with the aqueous solution in line 152.

Water wash column 168 may be of any suitable design. Typically, the water wash column operates with a recycling water loop, often with the recycle being at least about 20, say between about 50 and 500, mass percent of the crude biodiesel being fed to the column. A purge is taken from the loop via line 172. The purge balances the amount of water (aqueous phase) being provided via line 170. The purge is usually at a rate of between about 1 and 50, say 5 and 20, mass percent per unit time of the recycle rate in the loop.

The washing of the biodiesel is not critical to the broad processes of this invention. Where washing is used, any suitable sequencing of one or more wash steps may be used. For instance as an alternative, the crude biodiesel may be neutralized, but without significant conversion of soaps to free fatty acids, and water washed to remove soaps, then washed with an acidic aqueous medium, followed by a water wash to remove any residual acid from the wash with the acidic aqueous medium.

A washed biodiesel stream is withdrawn from washing column 168 via line 174 and is passed to drier 176 to remove water and residual methanol which exhaust via line 178. Drier 176 may be of any suitable design such as stripper, wiped film evaporator, falling film evaporator, and solid sorbent. Generally the temperature of drying is between about 80° C. and 220° C., say, about 100° C. and 180° C. The dried biodiesel is withdrawn as product via line 180. The biodiesel product contains free fatty acid and preferably has a free fatty acid content of less than about 0.8, and more preferably less than about 0.5, mass percent.

Returning to line 164, the aqueous phase from separator 162 is passed to evaporator 182 which provides a lower boiling fraction and a higher boiling fraction. While an evaporator may be used, it is also possible to use a packed or trayed distillation column with or without reflux. Generally the bottoms temperature of evaporator 182 is less than about 150° C., preferably between about 120° C. and 150° C. The distillation may be at any suitable pressure. A membrane separation system may, alternatively or in combination, be used with evaporator 182 to effect the sought concentration of the spent water. The bottoms fraction from evaporator 182 is removed via line 184. As it contains glycerin, it can be combined with the glycerin layer from lines 124 and/or 138 for further processing or disposal.

As shown, the separated glycerin-containing streams in lines 124 and 138 are combined and passed to column 186 where water and methanol are stripped from the glycerin. The lights exit via line 188 and may be further processed to remove water with the methanol being recycled. A glycerine-containing stream is removed via line 190. A portion of the glycerin-containing stream is passed via line 192 to catalyst reactor 194. Basic metal compound, which for the purposes of discussion, is an aqueous potassium hydroxide solution, is provided via line 196 to reactor 194. Catalyst reactor 194 is adapted to continuously remove water by distillation, and the water vapor is removed via line 198. The catalyst product, a potassium salt of glycerin is obtained from reactor 194 and passed via line 118 to transesterification reactor 110.

It is claimed:

1. A process for the base catalysis of glycerides to form lower alkyl esters comprising contacting said glyceride with lower alkanol under base catalyzed transesterification conditions comprising the presence of a catalytically effective amount of base catalyst comprising basic metal salt of glycerin to form monoalkyl ester and glycerin.

2. The process of claim 1 wherein the basic metal comprises one or more of alkali metals and alkaline earth metals.

3. The process of claim 2 wherein the basic metal comprises at least one of sodium, potassium and calcium.

4. The process of claim 1 wherein the base catalyst contains less than about 0.2 mole of water per mole of glycerin salt.

5. A process for the base catalysis of glycerides to form lower alkyl esters comprising:
a. contacting glyceride with lower alkanol under base catalyzed transesterification conditions comprising the presence of a catalytically effective amount of base catalyst to form monoalkyl ester and glycerin;
b. separating monoalkyl ester and glycerol to provide a monoalkyl ester fraction and a glycerin fraction;
c. in a separate step, contacting at least a portion of the glycerin fraction with at least one basic metal compound with glycerin and removing by vaporization water to provide a basic metal salt of glycerin; and
d. using at least a portion of the basic metal salt of glycerin to provide at least a portion of the base catalyst for step a.

6. The process of claim 5 wherein the basic metal compound comprises at least one of alkali and alkaline earth metal compound.

7. The process of claim 6 wherein vaporization of water occurs during the reaction to form the basic metal salt of glycerin.

8. The process of claim 7 wherein the glycerin salt is contacted with at least one lower alkanol to form basic metal lower alkoxide.

9. The process of claim 8 wherein the lower alkanol is at least one of methanol, ethanol, n-propanol, i-propanol, 1-butanol, isobutanol and 2-butanol.

10. A process for making basic transesterification catalyst comprising reacting at least one basic metal compound with glycerin to form metal salt of glycerol and removing by vaporization water.

11. The process of claim 10 wherein the catalyst contains less than about 0.2 mole of water per mole of glycerin salt.

12. The process of claim 11 wherein the basic metal compound comprises at least one of alkali and alkaline earth metal compound.

13. The process of claim 12 wherein the basic metal compound comprises at least one of basic metal oxide, basic metal carbonate, basic metal bicarbonate and basic metal hydroxide.

14. The process of claim 12 wherein vaporization of water occurs during the reaction to form the glycerin salt.

15. The process of claim 12 wherein the glycerin salt is contacted with at least one lower alkanol to form basic metal lower alkoxide.

16. The process of claim 15 wherein the lower alkanol is at least one of methanol, ethanol, n-propanol, i-propanol, 1-butanol, isobutanol and 2-butanol.

17. A transalkylation catalyst comprising basic metal salt of glycerin.

18. The catalyst of claim 17 which contains less than about 0.05 mole of water per mole of glycerin salt.

19. A transalkylation catalyst comprising at least about 5 mass percent lower alkoxide of basic metal and glycerin wherein the mole ratio of glycerin to lower alkoxide is less than about 5:1.

20. The transalkylation catalyst of claim 19 which contains less than about 0.05 mole of water per mole of lower alkoxide of basic metal.
